(19) United States
(12) Patent Application Publication
(10) Pub. No.: US 2021/0177544 A1
(43) Pub. Date: Jun. 17, 2021

(54) DENTAL PROSTHESIS

(71) Applicants: DENTSPLY SIRONA Inc., York, PA (US); DEGU-DENT GMBH, Hanau (DE)

(72) Inventor: Markus VOLLMANN, Gelnhausen (DE)

(73) Assignee: DENTSPLY SIRONA Inc., York, PA (US)

(21) Appl. No.: 17/267,875

(22) PCT Filed: Jul. 31, 2019
(86) PCT No.: PCT/EP2019/070678

§ 371 Date: Feb. 11, 2021

(30) Foreign Application Priority Data
Aug. 14, 2018 (EP) 18189013.8

(51) Int. Cl. A61C 5/73; A61C 13/00; A61K 6/833

(52) U.S. Cl. CPC A61C 5/73; A61K 6/833; A61C 13/0022

(57) ABSTRACT

The present invention is related to a dental prosthesis, in particular a crown, consisting of or comprising an inner primary part, such as a cap, which is joined in a fixed manner to an outer secondary part.

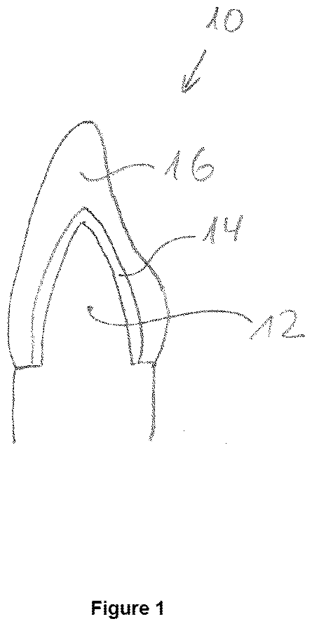

Further, the present invention is also directed to a dental kit for manufacturing or producing such a dental prosthesis, in particular a crown.

### FIELD OF THE INVENTION

The present invention relates to a dental prosthesis, in particular a crown, consisting of or comprising an inner primary part, such as a cap, that is connected in a fixed manner to an outer secondary part, such as a veneer.

The invention further relates to a dental kit for manufacturing or producing a dental prosthesis.

### BACKGROUND OF THE INVENTION

Usual dental prostheses, in particular crowns, consist of a framework of a metal as the primary part and a veneer covering the framework as the secondary part. This may be of ceramic. The material characteristics of the primary part and the secondary part are such that the modulus of elasticity of the primary part is greater than that of the secondary part.

Dental frameworks of zirconium oxide, aluminum oxide, zirconium mixed oxide, aluminum mixed oxide or combinations thereof are also known (DE 103 69 319 C5). The dental framework can be coated by hot-pressing a lithium silicate blank onto the framework.

### Objective of the Present Invention

An object underlying the present invention is to further develop a dental prosthesis of the type described above such that it has characteristics that correspond to those of a natural tooth.

A further object is to further develop a dental kit of the type described above such that the manufactured or produced dental prosthesis has characteristics that correspond to those of a natural tooth.

### DETAILED DESCRIPTION OF THE INVENTION

The object of the invention is substantially achieved in that the primary part consists of a plastic material or comprises such a material, that the secondary part comprises of a ceramic material or contains such a material, and that the primary part consists of material that has a modulus of elasticity EP and the secondary part consists of material that has a modulus of elasticity ES where EP<ES.

In particular, the modulus of elasticity EP of the primary part is in the range 5 GPa to 30 GPa, particularly preferably in the range 10 GPa to 25 GPa. The secondary part, with the higher modulus of elasticity ES in particular has a value in the range 40 GPa to 120 GPa, and preferably in the range 50 GPa to 75 GPa.

In accordance with the invention and by way of departure from the known prior art, wherein the primary part has a greater modulus of elasticity than the secondary part, a biomimetic dental prosthesis is provided which thus has the characteristics of a natural tooth.

It is here in particular provided that the primary part consists of or comprises at least one material from the group polymethyl methacrylate (PMMA), in particular highly-filled PMMA, polyether ether ketone (PEEK), triethylene glycol dimethacrylate (TEGDMA), diethylene glycol dimethacrylate (DEGMA), urethane dimethacrylate (UDMA) or a combination or part-combination thereof.

It is also possible to use one or more of the above-mentioned materials as the primary material, to which one or more ceramic fillers such as quartz (SiO2) or other glass ceramic fillers or oxide ceramic fillers have been added.

The primary part can also have a skeleton-like structure, for instance of a ceramic that is infiltrated with polymers.

A lithium silicate glass ceramic which is favored in particular is one which contains lithium disilicate and/or lithium metasilicate as the crystal phases.

Other ceramics, such as those based on phlogopite or glass ceramics from the LAS group (lithium oxide, aluminum oxide and/or silicon dioxide) or glass ceramics with virgilite crystals and/or sogdianite crystals may also be used.

The secondary part consists of or comprises a ceramic material, in particular a material from the group zirconium dioxide, in particular yttrium-stabilized zirconium dioxide, aluminum oxide, feldspar, lithium silicate glass ceramic or a mixture of one or more of these materials.

The secondary part advantageously consists of or includes as its starting material the following in % by weight:

| | |
|---|---|
| SiO₂ | 46.0-72.0 |
| Li₂O | 10.0-25.0 |
| ZrO₂ | 8.0-20.0 |
| Al₂O₃ | 0.1-8.0 |
| K₂O | 0.1-5.0 |
| CeO₂ | 0.0-4.0 |
| B₂O₃ | 0.0-4.0 |
| Na₂O | 0.0-4.0 |
| Tb₄O₇ | 0.0-2.5 |

at least one nucleating agent 1.0-10.0, such as P₂O₅,

as well as 0.0 to 4.0 of at least one additive,

where the entire sum is 100% by weight.

The secondary part preferably has a starting composition of, or includes, the following in % by weight:

| | |
|---|---|
| SiO₂ | 58-60 |
| Li₂O | 13.5-20.5 |
| ZrO₂ | 9.0-12.5 |
| P₂O₅ | 3.0-7.5 |
| Al₂O₃ | 0.5-6.0 |
| K₂O | 0.5-3.5 |
| CeO₂ | 0.5-2.5 |
| B₂O₃ | 0-3 |
| Na₂O | 0-3 |
| Tb₄O₇ | 0-1.5. |

The primary part can be manufactured from one block of plastic material in a CAD/CAM process. However, a thermoforming or deep drawing process is also possible.

The secondary part too can be manufactured in a CAD/CAM process. Other suitable manufacturing processes that are used to manufacture dental prostheses, such as pressing or additive manufacturing processes, can also be used.

In particular, the secondary part is connected to the primary part by bonding with adhesive or by cementing.

The usual dental polymer-based adhesives can be used as materials for the adhesive. These include non-curing and/or self-curing full adhesives or self-adhesives. Examples include dimethacrylate resins or bis-GMA (bisphenol A-glycidyl methacrylate).

Subject matter of the invention is also a dental kit for manufacturing or producing a dental prosthesis, in particular a crown, consisting of or comprising an inner primary part, such as a cap, which may be joined in a fixed manner to an outer secondary part, wherein the dental kit comprises:
a) a first block for forming, using a CAD/CAM process, an inner primary part, wherein the first block consists of or comprises plastic material, where the material of the first block has a modulus of elasticity Ep;
b) a second block for forming, using a CAD/CAM process, an outer secondary part, wherein the second block consists of or comprises ceramic material, where the material of the second block has a modulus of elasticity Es where Ep<Es; and
c) optionally, one or more adhesive bonding agents suitable for joining the inner primary part in a fixed manner to the outer secondary part.

In particular, the modulus of elasticity EP of the first block is in the range 5 GPa to 30 GPa, particularly preferably in the range 10 GPa to 25 GPa. The second block, with the higher modulus of elasticity ES in particular has a value in the range 40 GPa to 120 GPa, and preferably in the range 50 GPa to 75 GPa.

It is here in particular provided that the first block consists of or includes at least one material from the group polymethyl methacrylate (PMMA), in particular highly-filled PMMA, polyether ether ketone (PEEK), triethylene glycol dimethacrylate (TEGDMA), diethylene glycol dimethacrylate (DEGMA), urethane dimethacrylate (UDMA) or a combination or part-combination thereof.

It is also possible to use one or more of the above-mentioned materials for the first block, to which one or more ceramic fillers such as quartz (SiO2) or other glass ceramic fillers or oxide ceramic fillers have been added.

A lithium silicate glass ceramic which is favored in particular is one which contains lithium disilicate and/or lithium metasilicate as the crystal phases.

Other ceramics, such as those based on phlogopite or glass ceramics from the LAS group (lithium oxide, aluminum oxide and/or silicon dioxide) or glass ceramics with virgilite crystals and/or sogdianite crystals may also be used.

The second block consists of or includes a ceramic material, in particular a material from the group zirconium dioxide, in particular yttrium-stabilized zirconium dioxide, aluminum oxide, feldspar, lithium silicate glass ceramic or a mixture of one or more of these materials.

The second block advantageously consists of or comprises as its starting material the following in % by weight:

| | |
|---|---|
| SiO₂ | 46.0-72.0 |
| Li₂O | 10.0-25.0 |
| ZrO₂ | 8.0-20.0 |
| Al₂O₃ | 0.1-8.0 |
| K₂O | 0.1-5.0 |
| CeO₂ | 0.0-4.0 |
| B₂O₃ | 0.0-4.0 |
| Na₂O | 0.0-4.0 |
| Tb₄O₇ | 0.0-2.5 |

at least one nucleating agent 1.0-10.0, such as P₂O₅,

as well as 0.0 to 4.0 of at least one additive,

where the entire sum is 100% by weight.

The second block preferably has a starting composition of, or includes, the following in % by weight:

| | |
|---|---|
| SiO₂ | 58-60 |
| Li₂O | 13.5-20.5 |
| ZrO₂ | 9.0-12.5 |
| P₂O₅ | 3.0-7.5 |
| Al₂O₃ | 0.5-6.0 |
| K₂O | 0.5-3.5 |
| CeO₂ | 0.5-2.5 |
| B₂O₃ | 0-3 |
| Na₂O | 0-3 |
| Tb₄O₇ | 0-1.5. |

Further details, advantages and characteristics of the invention are provided not just from the claims and the characteristics to be drawn therefrom—singly and/or in combination—, but also from the following description of an embodiment shown in the drawing, as well as an example.

The teaching according to the invention is explained with reference to a crown 10, as shown in the single FIGURE, although no restriction results from this. Rather, the invention generally applies to any dental prosthesis that consists of a primary and secondary part, i.e. in the case of a crown a lower and an upper crown. Further examples include partial crowns, inlays, onlays, veneers and bridges, especially bridges that have three elements.

The FIGURE shows a prepared tooth stump 12. Taking the external shape of the tooth stump 12 into consideration, a cap 14 is first manufactured as the primary part and consists of or comprises plastic. Preferably the primary part consists of plastic. Preferred materials are high-density PPMA, UDMA, PEEK, DEGMA or TEGDMA or a mixture of one or more of these materials. Corresponding plastics with fill additives such as SiO2 can also be used. The modulus of elasticity of the material used lies in the range 5 to 30 GPa.

The cap 14 can be manufactured from a plastic block using a CAD/CAM process, without any restriction being intended by this.

After manufacture of the cap 14, taking into consideration its outer geometry, the teeth adjoining the tooth stump and the antagonists, a veneer 16 is manufactured as secondary part in a CAD/CAM process from a block of lithium silicate glass ceramic or another suitable ceramic material that has a modulus of elasticity between 40 and 120 GPa. After cementing of the cap 14 to the stump 12, the secondary part 16 is bonded to the primary part. It can also be cemented.

By way of departure from a usual dental prosthesis, which consists of a framework of metal and a veneer, a biomimetic dental prosthesis is provided, in which the modulus of elasticity of the primary part corresponds to that of the dentine material and that of the secondary part corresponds to that of the dental enamel material, where the strength may be selected to be higher if necessary.

To permit conclusions about the working life of a corresponding dental prosthesis, artificial ageing was performed with a chewing simulator (Chewing Simulator CS-4.8 with TC4, made by SDM Mechatronic GmbH, Feldkirchen, Germany). For this purpose, ten crowns were manufactured, with the material for the primary part being highly-filled PMMA with a modulus of elasticity of 13 GPa. The primary part was manufactured from a PMMA block in a CAD/CAM process.

The secondary part was manufactured from a lithium disilicate glass ceramic block in a CAD/CAM process. The material used was a lithium disilicate glass ceramic marketed under the name CELTRA Duo by DeguDent GmbH, Hanau, Germany.

The crowns so manufactured were bonded to metal stumps with an adhesive material marketed under the name Multilink Hybrid Abutment. The ten crowns were each subjected to 1.2 million chewing cycles (1 Hz) together with 6,000 thermal cycles at 5° C./150° C. in water. Following chewing simulation, there were no cracks, fissures or delaminations detected. The mean breaking load of the ten crowns after ageing was 4,437 N+/−1,012 N.

1. A dental prosthesis, comprising an inner primary part, which is joined in a fixed manner to an outer secondary part,
wherein the inner primary part includes plastic material, and the outer secondary part includes ceramic material, and
wherein the plastic material has a modulus of elasticity E_{P} and the ceramic material has a modulus of elasticity E_{S} where E_{P}<E_{S};
wherein the modulus of elasticity E_{P} of the inner primary part is 5 GPa≤E_{P}≤30 GPa, and/or the modulus of elasticity E_{S} of the outer secondary part is 40 GPa≤E_{S}≤120 GPa;
wherein the inner primary part includes at least one material being selected from the group consisting of PMMA (polymethyl methacrylate), PEEK (polyether ether ketone), TEGDMA (triethylene glycol dimethacrylate), DEGMA (diethylene glycol dimethacrylate), UDMA (urethane dimethacrylate) and a combination or part-combination thereof; and
wherein the outer secondary part includes a material being selected from the group consisting of zirconium dioxide, aluminum oxide, feldspar, lithium silicate glass ceramic or mixtures and part-mixtures thereof.

2. The dental prosthesis according to claim 1, wherein the modulus of elasticity E_{P} of the inner primary part is 10 GPa≤E_{P}≤25 GPa, and/or the modulus of elasticity E_{S} of the outer secondary part is 50 GPa≤E_{S}≤75 GPa.

3. The dental prosthesis according to claim 1, wherein the inner primary part includes at least one material to which a ceramic-based fill material being selected from the group consisting of SiO₂, feldspar and zirconium dioxide has been additionally added.

4. The dental prosthesis according to claim 1, wherein the inner primary part has a skeleton structure infiltrated with at least one polymer, said structure includes a ceramic material.

5. The dental prosthesis according to claim 1, wherein the inner primary part is connected to the outer secondary part by bonding with adhesive or by cementing.

6. The dental prosthesis according to claim 1, wherein the outer secondary part includes a starting material with the following composition in % by weight:

| | |
|---|---|
| SiO₂ | 46.0-72.0 |
| Li₂O | 10.0-25.0 |
| ZrO₂ | 8.0-20.0 |
| Al₂O₃ | 0.1-8.0 |
| K₂O | 0.1-5.0 |
| CeO₂ | 0.0-4.0 |
| B₂O₃ | 0.0-4.0 |
| Na₂O | 0.0-4.0 |
| Tb₄O₇ | 0.0-2.5 |

at least one nucleating agent 1.0-10.0,
0.0 to 4.0 of at least one additive,
where the entire sum is 100% by weight.

7. The dental prosthesis according to claim 1, wherein the outer secondary part includes a starting material with the following composition in % by weight

| | |
|---|---|
| SiO₂ | 58-60 |
| Li₂O | 13.5-20.5 |
| ZrO₂ | 9.0-12.5 |
| P₂O₅ | 3.0-7.5 |
| Al₂O₃ | 0.5-6.0 |
| K₂O | 0.5-3.5 |
| CeO₂ | 0.5-2.5 |
| B₂O₃ | 0-3 |
| Na₂O | 0-3 |
| Tb₄O₇ | 0-1.5. |

8. A dental kit for manufacturing or producing a dental prosthesis, comprising an inner primary part, which is joined in a fixed manner to an outer secondary part, wherein the dental kit comprises:
a) a first block for forming, an inner primary part, wherein the first block includes plastic material, where the plastic material of the first block has a modulus of elasticity Ep;
b) a second block for forming, an outer secondary part, wherein the second block includes ceramic material, where the ceramic material of the second block has a modulus of elasticity Es where Ep<Es; and
c) optionally, one or more adhesive bonding agents suitable for joining the inner primary part in a fixed manner to the outer secondary part;
wherein the modulus of elasticity E_{P} of the plastic material of the first block is 5 GPa≤E_{P}≤30 GPa, and/or the modulus of elasticity E_{S} of the ceramic material of the second block is 40 GPa≤E_{S}≤120 GPa;
wherein the first block includes at least one material being selected from the group consisting of PMMA (polymethyl methacrylate), PEEK (polyether ether ketone), TEGDMA (triethylene glycol dimethacrylate), DEGMA (diethylene glycol dimethacrylate), UDMA (urethane dimethacrylate) and a combination or part-combination thereof; and
wherein the outer secondary part includes a material being selected from the group consisting of zirconium dioxide, aluminum oxide, feldspar, lithium silicate glass ceramic and mixtures or part-mixtures thereof.

9. The dental kit according to claim 8, wherein the modulus of elasticity E_{P} of the plastic material of the first block is 10 GPa≤E_{P}≤25 GPa, and/or the modulus of elasticity E_{S} of the ceramic material of the second block is 50 GPa≤E_{S}≤75 GPa.

10. The dental kit according to claim 8, wherein the first block includes at least one material to which a ceramic-based fill material being selected from the group consisting of SiO₂, feldspar and zirconium dioxide has been additionally added.

11. The dental kit according to claim 8, wherein the second block includes a starting material with the following composition in % by weight:

| | |
|---|---|
| SiO₂ | 46.0-72.0 |
| Li₂O | 10.0-25.0 |
| ZrO₂ | 8.0-20.0 |
| Al₂O₃ | 0.1-8.0 |
| K₂O | 0.1-5.0 |
| CeO₂ | 0.0-4.0 |
| B₂O₃ | 0.0-4.0 |
| Na₂O | 0.0-4.0 |
| Tb₄O₇ | 0.0-2.5 |

at least one nucleating agent 1.0-10.0,
0.0 to 4.0 of at least one additive,
where the entire sum is 100% by weight.

12. The dental kit according to claim 8, wherein the second block includes a starting material with the following composition in % by weight:

| | |
|---|---|
| SiO₂ | 58-60 |
| Li₂O | 13.5-20.5 |
| ZrO₂ | 9.0-12.5 |
| P₂O₅ | 3.0-7.5 |
| Al₂O₃ | 0.5-6.0 |
| K₂O | 0.5-3.5 |
| CeO₂ | 0.5-2.5 |
| B₂O₃ | 0-3 |
| Na₂O | 0-3 |
| Tb₄O₇ | 0-1.5. |

13. The dental prosthesis according to claim 5, wherein the adhesive material includes at least one material being selected from the group consisting of dental polymer-based adhesives.

14. The dental prosthesis according to claim 6, wherein at least one nucleating agent is P₂O₅.

15. The dental kit according to claim 8, further comprising one or more adhesive bonding agents suitable for joining the inner primary part in a fixed manner to the outer secondary part.

16. The dental prosthesis according to claim 11, wherein at least one nucleating agent is P₂O₅.
